(19) United States
(12) Patent Application Publication
(10) Pub. No.: US 2011/0263995 A1
(43) Pub. Date: Oct. 27, 2011

(54) Comprehensive Myocardial Repolarization Capture Wave-Format Method

(76) Inventor: Guangren Chen, Palm Harboor, FL (US)

(21) Appl. No.: 12/764,146

(22) Filed: Apr. 21, 2010

(51) Int. Cl. A61B 5/0402

(52) U.S. Cl. 600/515

(57) ABSTRACT

A method of displaying a cardiac cycle of a heart into a three set colorable waveform comprising a plurality electrocardiography data taken over a period of time, said cardiac cycle information comprising a P wave, a PR segment, a QRS complex, and a ST segment, the method comprising generating a first wave hump by recalculating said P wave by using the Time-Frequency domain to determine an influxes of data compensation; generating a second wave hump by recalculating said PR segment by using integral formulaic expressions; generating a third wave hump by recalculating said QRS complex and said ST segment by using Hilbert space in the inner product.

### CROSS REFERENCE TO RELATED APPLICATION

### Field of the Invention

This invention is in the field of monitoring and diagnosing electrical activity within the human body.

### Background

This application has to demonstrate the prior art, for example, in the heart, electrical signals coordinate the rhythmic pumping of the cardiac muscles and the bio-potential signals resulting from the heart's electrical activity are routinely recorded. This recordation of the well-coordinated electrical events that take place within the heart is called an electrocardiogram (ECG). There are many different types of ECG. However, rhythmic ECG signals with respect to various time and amplitude and frequency content are obtained from different regions of the heart. These regions around the organ include the conduct bundle and myocardium that generate various electrical activity bio-electric signals that travel throughout the human body surface. The purpose of this ‘Enhance Dynamic Flow Data’ (hereafter referred to as EDFD) electrode signal transmission is to distribute information from extra high impedance when a necessary function is being carried out. For example, body surface electrical phenomena currency exhibits only about 10% of the result of this electro-physiological activity in the presence of various bio-electric voltages. If using EDFD electrode a system may be able to obtain good ECG recordings. Clinically, the ECG technique is currently used to diagnose a number of physiological conditions. The status of heart muscles (e.g., potential ischemia) is not often detected and various life-threatening heart arrhythmias and ischemia are not routinely identified. The reason for non-identification is ischemia detection is in “T” section of ultra low frequency, and existing ECG systems are not sensitive enough for this information; however, if detected, the information could also help for more signal and guide specific testing modalities of the patient. Currently, there is a company that provides a mi-ECG instrument for medical research and clinical practice. Use of the ECG is especially widespread and the equipment is highly advanced for ischemia diagnosis. There is a sophisticated ischemic diagnostic ECG instrument, and ischemia monitoring devices for routine use for a variety of medical environments and even portable devices. In spite of the sophisticated ECG systems, the missing generated data still has a lot of promising information that has not been exploited to its maximum potentialities, and current system missing this generated data may not make a proper diagnosis of a heart condition. With little training, most medical staff cannot obtain a good deal of information from the ECG signals. Automated, computer analysis is a very accurate assistant to the Cardiologist in diagnosing various heart abnormalities. Because of the nature and convenience of obtaining and interpreting the ECG signals, all ECG systems are very different, but almost every patient in an operating room (OR), intensive care unit (ICU), or ER environment is routinely monitored with an ECG equipment, so the waveform settlement are very important. Therefore, it is highly desirable in the art to have a special electrode that provides a highly sensitive signal, micro-chip input that is indicative of the state of strip EDFD electrode. This input chip corresponds to a numeric value with respect to the heart activity to maximize heart's information activity, respectively.

Another shortcoming in the previous ECG systems are measuring the length of wires mainly related to the device and to patient, and then having these long wires tangled. The old ECG systems are used for routine patient monitoring, or screening normally obtained in the OR, ICU, or ER environment. When using the old ECG systems, the 12 leads ECG with 10 leads cables are inconvenient to use and to put on the patient. When monitoring or screening the patients, the lead cables must have good contact with the skin and must be monitored in a special environment. The one strip with six electrodes are relatively simple to attach by an adhesive patch and easy to remove. (i.e., usually the upper-body clothing must be removed). Sometimes during use, the leads often became disconnected or out-of-order that requires re-set up. Such difficulties cause the following problems. A trained technical staff personnel is normally required to set up the leads. Often the 10 lead cables are winding and tangled at all times; therefore, resulting in wrongful connections of the leads and not being able to obtain the desired EEG data. There is an additional cost associated with using trained staff, and the current medical reimbursement policies tend to encourage minimal use of trained staff in the hospital. The attachment protocol of the lead cables take a considerable amount of time especially when multiple electrodes are required. The ordeal is difficult and tedious for the patient. Furthermore, if a patient were to be relocated in a different area in the hospital, all electrodes must be detached or the entire unit must relocate with the patient. Therefore, it is highly necessary in the art to use a Bluetooth model or a wireless configuration between patient that is connected to leads and device that monitors and displays information.

In hospital use of Magnetic Resonance Imaging (hereafter referred to as MRI) machines have become a routine method for obtaining information regarding a patient's anatomy and physiology. Currently, however, not many patients are monitored with an ECG instruments while they are in the MRI. Basically, the MRI and ECG equipment are not compatible. The operating MRI produces strong radio frequency (RF) fields and large static magnetic fields are always present. These fields create a strong magnetic flux that induces current flow in electrodes and to any attached electrode wires that form a loop. The result of the increase in the magnet flux has been reported or a resulted in instances of localized skin burns from the electrodes and looped wires residing in MRI machines. Therefore, causing current distribution that is diffused throughout the patient's body. Such situations can be fatal if the current induced is sufficiently large. The presence of equipment near the MRI machine can also interfere with the diagnostic quality of the MRI images themselves by causing distortions in the MRI output. Also, the radio-frequency (RF) fields of the MRI machine can corrupt the weak signals being recorded by ECG equipment and especially even weaker signals associated with 12 lead ECG instruments. For this reason, a special screen room is built around the MRI machine to prevent from affecting other equipment in the vicinity of the imaging device. Generally, all patients in OR, ER, ICU, CCU, have to remove all unnecessary equipment, and must keep it outside the screen room. To solve these and other associated problems with prior art measuring with EDFD electrodes, it is an object of the present invention to provide a better ECG waveform that avoids a macro electronic countermeasure in hospital situation. It is an object of the present invention to provide easy set up for using EDFD electrode.

Existing ECG, a century old diagnostic tool, checking ST-T elevation and depression, cannot detect minute myocardial cellular activity associated with early problematic conditions. The rapid acquisition and signal processing techniques utilizing low and high frequency bands, massive data acquisition and storage methods, all necessary for the i-ECG system, have only been developed in the last ten years. As stated, this allows the interrogation of the entire myocardium, mapped in 3D and real time at the cellular level. This data is simultaneously converted into a two dimensional representation described above. The clinical procedure to obtain this vast amount of information is approximately 30 seconds after the surface electrodes and cables are attached.

### SUMMARY OF THE INVENTION

A non-transitory computer readable medium comprising a computer program to display a a colorable waveform wherein said colorable waveform is composed by a processor unit that is used to take into consideration the information from a set of lead cables; a recording unit stores the data from the set of lead cables on a memory device; a calculating unit that determines the waveform arrhythmia of a organ, wherein said waveform arrhythmia used to determine the oxygen supply and the organ strength; a display unit that is used to display the information on a display apparatus that is used to show the waveform arrhythmia, wherein the last of the waveform arrhythmia of the last cycle demonstrates a repolarization of the ventricles; a colorable unit that is used to determine the hue of the waveform arrhythmia by using Hilbert space in the inner product; and the processor unit uses the data from the memory device and sends the information to the calculating unit to determine the strength and oxygen supply to be computed by colorable unit that is used to determine the hue of the waveform and a projects the information on the display unit.

In another exemplary way, a method of displaying a cardiac cycle of a heart into a colorable waveform comprising a plurality electrocardiography data taken over a period of time, said cardiac cycle information comprising a P wave, a PR segment, a QRS complex, and a ST segment, the method comprising generating a first wave hump by recalculating said P wave by using the Time-Frequency domain to determine an influxes of data compensation, generating a second wave hump by recalculating said PR segment by using integral formulaic expressions, generating a third wave hump by recalculating said QRS complex and said ST segment by using Hilbert space in the inner product.

The method of displaying the cardiac cycle further recording the data on a non-transitory computer readable medium that is used to compare new colorable waveforms with old colorable waveforms.

### DESCRIPTION OF THE DRAWINGS

FIG. 1 is the overall process of receive the information of the human organ.

FIG. 2 is specific single cycle cardiac arrhythmia of the human heart segmenting of each specific period.

FIG. 3 is colorable wave display of the cycles of cardiac arrhythmia.

### DETAILED DESCRIPTION

The following detailed description is provided to assist the reader in gaining a comprehensive understanding of the methods, apparatuses and/or systems described herein. Accordingly, various changes, modifications, and equivalents of the systems, apparatuses, and/or methods described herein will likely suggest themselves to those of ordinary skill in the art. Also, descriptions of well-known functions and constructions are omitted to increase clarity and conciseness.

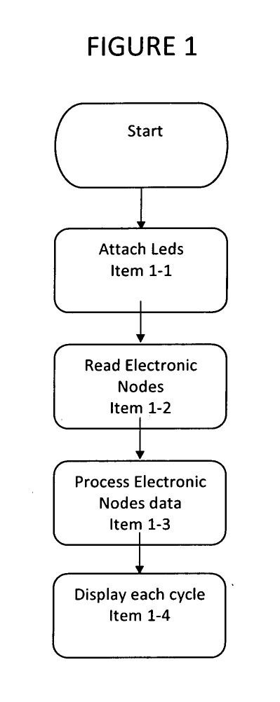

With regards to FIG. 1, the system is used by attaching the lead cables to the individual 1-1. The system is attached, the system will start with a predetermined time period to read the signals that are generated by the human organ 1-2. The MyVisto will start to process the electrical signals that are received from the lead cables 1-3. Thereafter, the system will display on the computer screen the information that is generated by the lead claims 1-4. The information being displayed is used by medical personal to determine irregularities in the heart rhythm.

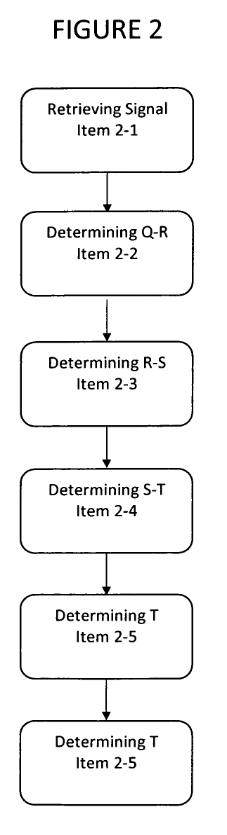

FIG. 2 displays the different steps of determining the signal information. The system will retrieve the signal 2-1. The signal determination is done by first determining the Q-R 2-2, then determining R-S 2-3, determining S-T, and last determining the T 2-5. After determine each of the regions, the system will display all the information on a display apparatus. The information is displayed to allow a medical personal to determine specifically the different regions of the organ and color scale information.

Typical ECG tracing of the cardiac cycle (heartbeat) consists of a P wave, a QRS complex, a T wave, and a U wave which is normally visible in 50% to 75% of ECGs. The baseline voltage of the electrocardiogram is known as the isoelectric line. Typically the isoelectric line is measured as the portion of the tracing following the T wave and preceding the next P wave. The T wave represents the repolarization (or recovery) of the ventricles. The interval from the beginning of the QRS complex to the apex of the T wave is referred to as the absolute refractory period. The last half of the T wave is referred to as the relative refractory period (or vulnerable period). The ECG waveform is linear sine waveform. As these methods are found, devices are being created that monitor the heart in order to detect the onset of dangerous rhythms and to correct them before they cause death.

Research being conducted suggests that a crescendo in ECG heterogeneity, both in the R-wave and the T-wave, often signals the start of ventricular fibrillation. In patients with coronary artery disease, exercise increases T-wave heterogeneity, but this effect is not seen in normal patients. These results, when combined with other pieces of emerging evidence, suggest that R-wave and T-wave heterogeneity both have predictive value.

Every heart beat contains a bundle of electrophysiological conducting energy. Each heart beat goes through the following process of atrioventricular node, sinus node, then to left and right bundle branch, into the purkinje fibers, and into the myocardial where about 99% of “arrhythmia disease” is determined. One major categories of cardiac disease can be displayed in the time domain. The myocardial part conduction is depended on the blood volume and cardiomyocytes inner conduction. Also “cardiacvascular disease” is another major category of cardiac disease, the signal changes in spatial and temporal domain with vector that causes the time domain to be deeply flawed.

This method uses a wavelet analysis as compared to the original time-domain ECG signal. It uses a time-frequency domain analysis. It shows ECG in the time-frequency domain of the energy distribution, and from the time domain, frequency domain, energy domain of the ECG signal feature extraction, and then in combination with other information to make an accurate diagnosis of the condition. One aspect of the present method is it the depolarization of the myocardium, which is represented by the QRS complex. Another aspect of the present invention is it measures the repolarization of the myocardium, which is represented by the T wave. In the preferred embodiment of the present invention, the ECG signal is transformed using signal processing techniques to generate a two-dimensional time-frequency map of the signal, which represents the energy distribution of the ECG signal in both the time and the frequency domains during a single beat or multiple beats of the myocardium, to facilitate the detection and analysis of any energy imbalance associated with the repolarization of the myocardium.

In the Wavelet analysis the window area is fixed, but the time window and frequency window can change. From another point of view, wavelet function actually is a series of band-pass or low-pass filter through the window function. The translation and contraction of the heart produce a series of transformations of different center frequency, bandwidth of different filters.

Wavelet transformation is a time-frequency localization signal analysis method, in which a signal is viewed through a window with a certain width. The window function defines the window width, which is varied to achieve (a) a higher frequency resolution (but lower time resolution) of the low frequency components of a signal, and (b) a higher time resolution (but lower frequency resolution) of the high frequency components of the signal.

As applied to an ECG signal, increasing resolution in the time domain improves the accuracy of measuring the QRS complex, because the QRS complex occurs over a very short time period. Conversely, increasing resolution in the frequency domain improves the accuracy of measuring the T wave, as the component frequencies of the T wave are in the lower frequency ranges of the ECG signal.

The signal processing of the ECG signal via wavelet transformation according to one aspect of the invention is described below in further detail. Here, continuous wavelet transform is used for multi-domain analysis of the ECG signal to generate a representation of the energy distribution of the ECG signal in both the time and the frequency domains.

In a continuous wavelet transform the basic wavelet shall be produced after the displacement of b, then at different scales of a signal to be analyzed under the inner product.

In the above definition, the basic wavelet must satisfy certain constraints, in the whole timeline of the integral value must be 0; the absolute value of the integral in the whole timeline must be less than infinity. In addition, signal transform but also to satisfy a certain “stability conditions.”

In the instrument, the signal processing uses wavelet analysis of ECG. Select the scale parameter a, put it with the original signal to begin the period of comparison, from the mathematical point of view, is through convolution, comparative relevance (correlation). The wavelet coefficients are calculated at this point. The coefficients expressed is a signal and the wavelet scale a of relevant correlation. The greater the Wavelet coefficients the more similar the correlation is. This result depends on the choice of the base wavelet shape. The first step in wavelet transformation is the construction of an appropriate wavelet base, on which all wavelet analysis windows are based, as all of such windows are scaled and/or shifted versions of the wavelet base. Commonly used wavelet bases may include the Mexican Hat wavelet, the Morlet wavelet, the Coif wavelet, the dbN wavelet, the symN wavelet, the Bior wavelet, etc. The selection or construction of suitable wavelet base may be made according to support length, symmetry, vanishing matrix, regularity and similarity principles.

The wavelet shifts right, this achieved by increasing the translation parameter b value (b=b+1). Repeat the first and the second step, until the processing of the signal reaches the tail. Increase the wavelet scale parameter a (a=a+1), then repeat first to the third steps. A continuous wavelet transform will be the final result when the required full-scale parameters and translation parameters of the calculation are completed.

After the completion of the analysis a is the vertical axis, b the horizontal axis, the encoded wavelet coefficients, namely the ECG time with frequency domain of the 2D waveform image energy distribution is displayed. From this 2D time-frequency domain energy distribution relevant characteristics can be extracted, combined with pathology and a more in-depth knowledge of signal processing knowledge, to make the relevant condition diagnosis.

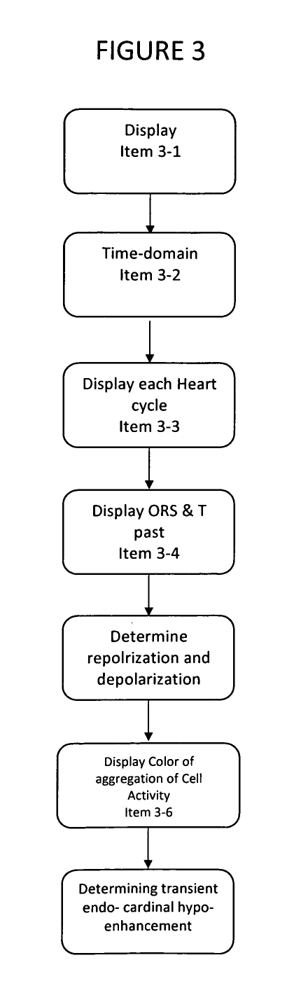

FIG. 3 displays the steps for displaying the organ signal. The organ signals are displayed by determining time-domain relative to the frequency domain. The display is determined by the using the time frequency domain, 3-2. The time-frequency domain is used to determine the frequency during a set time period to allow proper monitor of the organ. After the system has collected enough information within a set time period, the system will then display each heart cycle, item 3-3. The system will display the QRS & T, item 3-4. The system will then make a colorable waveform. The colorable waveform will display the amount of intensity of the repolarization and depolarization. The color level is determined below illustrated below.

The MyoVista instrument using wavelet transform and continuing wavelet transform as a time-frequency domain analysis tools. It is nonlinearly waveform, focused on spatial and temporal domain with beat-by-beat. (There are numerous methods can be applied to the analysis of time-frequency domain analysis) this is one of the methods the present invention uses:

For any function, ƒ(t)εL²(R), its continuous wavelet transform is defined as L² the Hilbert space in the inner product:

$$Wf ( a , b ) = {\vert a \vert}^{- \frac{1}{2}} {\int }_{- ∞}^{+ ∞} f ( t ) {ψ}^{*} ( \frac{t - b}{a} ) d t = f ( b ) * ( {\vert a \vert}^{- \frac{1}{2}} {ψ}^{*} ( \frac{- b}{a} ) )$$

W(a,b) Equivalent ƒ(t) with

$$\overline{ψ ( \frac{t - b}{a} )}$$

in the real axis R of t points of |a|^{-1/2} times. The Functions can also be written ƒ(t) and

$${\vert a \vert}^{- \frac{1}{2}} \overline{ψ ( \frac{t - b}{a} )}$$

in the form of convolution. In the integral area R interval for k paragraph divide, broken down Wₖ(a,b) equals to ƒ(t) and

$$\overline{ψ ( \frac{t - b}{a} )}$$

in the area [k,k+1] same interval on the t-fold integrals |a|^{-1/2} time. Wₖ(a,b) for integer k summation will be carried out, the formula will receive W(a,b) another form of expression. When k checks for a large enough value, it can be assumed tε[k,k+1] that in the interval, it is ƒ(t) equivalent to ƒ(k). Therefore, the reference points can be ƒ(t). The first points on

$$\overline{ψ ( \frac{t - b}{a} )}$$

in the [k,k+1] interval, multiplied by |a|^{-1/2} ƒ(k) the final summation of the integer k. The use of integral formula,

$$\overline{ψ ( \frac{t - b}{a} )}$$

in the range of [k,k+1] on t can be expressed as points

$$\overline{ψ ( \frac{t - b}{a} )}$$

in the interval (−∞,k+1] of t points

$$\overline{ψ ( \frac{t - b}{a} )}$$

in the interval (−∞,k] of t points of the difference. The difference be multiplied by |a|^{-1/2} ƒ(k), and then carried out on the integer k summation. This will be the W(a,b) ultimate form of the wavelet expression. Therefore, for any scaling factor a, from one value to get length(ƒ) (the original signal length) of the translation factor b; Wavelet coefficients W(a,b) can be shaped through the input signal ƒ(t) and ∫_{-∞}^{k}Ψ(t)dt for the formula for convolution. And then check the value of the margin ∫_{-∞}^{k}Ψ(t)dt which can be obtained through direct calculation or look-up table method.

Since self variable is time, which means horizontal axis is time, vertical axis is the signal changes. The dynamic signal x (t) is to describe the signal values pick at different time of functions. The display of the 2-Dimensional wavelet function is the second derivative of the Gauss function, and its time and frequency domain be defined as:

$$ψ ( t ) = ( 1 - {t}^{2} ) {e}^{- \frac{{t}^{2}}{2}}$$

$$ψ ( ω ) = \sqrt{2 π} {ω}^{2} {e}^{- \frac{{ω}^{2}}{2}}$$

To make a translation and expansion for a standard wavelet function (known as the base wavelet) results into a cluster function. The cluster function has two factors. The first function is the cluster which is the scaling factor ‘a’ and translation factor ‘b.’ This allows the system to make time and frequency analysis for ECG signals by using the continuous wavelet transform. That is, to make inner product on space L². Through variable substitution, the wavelet coefficients is W(a,b) expressed as input signal ƒ(t) with form like such as ∫_{-∞}^{k}ψ(t)dt of the formula convolution, integral interval is the real number axis.

Finally, the continuous wavelet transform coefficients will get the following results through use of the integral formulaic expressions:

$$W ( a , b ) = {\vert a \vert}^{- \frac{1}{2}} {\sum }_{k} f ( k ) ( {\int }_{- ∞}^{k + 1} \overline{ψ ( \frac{t - b}{a} )} d t - {\int }_{- ∞}^{k} \overline{ψ ( \frac{t - b}{a} )} d t )$$

Through the above-mentioned method the continuous wavelet transform is calculated by coefficients of input signal x(t), and then encoded to W(a,b). There are two ways to display. One is direct display and the other shows the absolute value. Since the square of the wavelet transform coefficients can express the distribution of energy, so the absolute value displayed represents the energy display. The encoding includes the line encoding and the integral encoding. It not only uses the absolute value to display, but also according to the integral encoding, take the absolute value after calculating the wavelet transform coefficients, and then transform according to certain rules, in order to fit the numerical color display. Finally use “a” as the vertical axis and “b” as the horizontal axis. Then use “a” and “b” as an encoded wavelet transform of coefficients to the displayed distribution maps (2D) of ECG signal convolution through time-frequency domain energy. The display of the wavelet helps to make diagnosis for some related illness by extracting the relevant characteristics from time-frequency domain energy distribution of ECG signals.

For signal analysis in time-domain, sometimes there will be numerous signals in time domain whose parameters are the same, but it does not explain the signal on exactly the same as a matter. Because the signal does not only change over time, but also over frequency, phase, spatial and other information related to, and this requires further analysis of the frequency of the signal structure and the signal in the frequency domain description within time domain.

Dynamic signal from time domain transforms to the frequency domain through Signal Processing series and Signal Processing Transform, periodic signal by Fourier series, non-periodic signal by Fourier transform. The traditional Fourier transform frequency domain analysis is one of the tools, but through the Fourier transform of the frequency-domain characteristics of the global in nature, we cannot do real-time reflect, that is, the time-domain information is lost and many signals are overlapped.

ECG is a non-stationary signal, time-domain characteristics cannot be discarded. And since the statistical characteristics of non-stationary signals that change over time, then the non-stationary signals of primary interest will be very naturally, in its local statistical properties, so the need to adopt time-frequency domain analysis tools in the time domain and frequency domain two pairs of ECG for feature analysis, we need to find useful information. The so-called time-frequency domain analysis, that is, the local properties of non-stationary signals in time domain and frequency domain on a two-dimensional co-expressed.

A number of exemplary embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

It is important to note that although embodiments of the present invention have been described in the context of a fully functional system, those skilled in the art will appreciate that the mechanism of the present invention and/or aspects thereof are capable of being distributed in the form of a computer readable medium of instructions in a variety of forms for execution on a processor, processors, or the like, and that the present invention applies equally regardless of the particular type of signal bearing media used to actually carry out the distribution. Examples of computer readable media include: nonvolatile, hard-coded type media such as read only memories (ROMs) or erasable, electrically programmable read only memories (EEPROMs), recordable type media such as floppy disks, hard disk drives and CD-ROMs, and transmission type media such as digital and analog communication links.

1. A non-transitory computer readable medium is comprising: computer program to display a colorable waveform wherein said colorable waveform is composed by:
a processor unit that is used to take into consideration the information from a set of lead cables;
a recording unit stores the data from the set of lead cables on a memory device;
a calculating unit that determines the waveform arrhythmia of a organ, wherein said waveform arrhythmia used to determine the oxygen supply and the organ strength;
a display unit that is used to display the information on a display apparatus that is used to show the waveform arrhythmia, wherein the last of the waveform arrhythmia of the last cycle demonstrates a repolarization of the ventricles;
a colorable unit that is used to determine the hue of the waveform arrhythmia by using Hilbert space in the inner product; and
the processor unit uses the data from the memory device and sends the information to the calculating unit to determine the strength and oxygen supply to be computed by colorable unit that is used to determine the hue of the waveform and a projects the information on the display unit.

2. A method of displaying a cardiac cycle of a heart into a three set colorable waveform comprising a plurality electrocardiography data taken over a period of time, said cardiac cycle information comprising a P wave, a PR segment, a QRS complex, and a ST segment, the method comprising:
generating a first wave hump by recalculating said P wave by using the Time-Frequency domain to determine an influxes of data compensation;
generating a second wave hump by recalculating said PR segment by using integral formulaic expressions; and
generating a third wave hump by recalculating said QRS complex and said ST segment by using Hilbert space in the inner product.

3. The method of displaying the cardiac cycle of claim 2 further comprising:
recording the data on a non-transitory computer readable medium that is used to compare new colorable waveforms with old colorable waveforms.

4. The method of displaying the cardiac cycle of claim 2 further comprising:
displaying the three dimensional cardiac cycle wherein the displaying illustrates a three set waves cycle.

5. The method of displaying the cardiac cycle of claim 4 further comprising:
displaying the three set waves by the integral formulaic expression
$$W ( a , b ) = {\vert a \vert}^{- \frac{1}{2}} {\sum }_{k} f ( k ) ( {\int }_{- ∞}^{k + 1} \overline{ψ ( \frac{t - b}{a} )} d t - {\int }_{- ∞}^{k} \overline{ψ ( \frac{t - b}{a} )} d t ) .$$
